(19) United States
(12) Patent Application Publication
(10) Pub. No.: US 2010/0051829 A1
(43) Pub. Date: Mar. 4, 2010

(54) Method for Putting Code Information on a Wafer Case

(75) Inventor: Yasue NISHIOKA, Tokyo (JP)

Correspondence Address: HOGAN & HARTSON L.L.P., 1999 AVENUE OF THE STARS, SUITE 1400, LOS ANGELES, CA, 90067, US

(73) Assignee: MIRAIAL CO., LTD., Tokyo (JP)

(21) Appl. No.: 12/202,996

(22) Filed: Sep. 2, 2008

(51) Int. Cl. A61N 5/00

(52) U.S. Cl. 250/492.2

(57) ABSTRACT

A laser light is irradiated on a wafer case made of polymer materials that have a high transparency to visible light and a low transparency to laser light having a wavelength other than the optical wavelength. The wafer case material in the irradiated portion foams, blackens, melts or evaporates by the irradiation. Thus, wafer case information can be marked by concavities and convexities or changing color formed on the wafer case surface. The wafer case information can be formed as a one-dimensional or two-dimensional code. Multiple laser lights can be irradiated on the same portion of the wafer case surface from different directions to the wafer case surface, and thus wafer case information is marked on only the neighborhood of the wafer case surface by foaming, blackening, melting or evaporating only the neighborhood of the wafer case surface that the laser light is irradiated on.

### CROSS-REFERENCE TO RELATED APPLICATIONS

This application claims the benefit of priority of U.S. provisional application No. 61/035,988, filed on Mar. 12, 2008.

### BACKGROUND OF THE INVENTION

1. Field of the Invention

This invention relates to a method for marking indicators of two-dimensional code on a transparent wafer container or case that stores and keeps semiconductor substrates, and to a wafer case on which two-dimensional code is marked using the method.

2. Description of Related Art

Silicon wafers and compound semiconductor wafers (hereinafter called “wafers” or “wafer”) are stored and kept in a wafer case in semiconductor factories, for example. The wafers may be handled or carried in a unit of a wafer case. Generally, marks are recorded on every wafer case as well as every wafer. The marks recorded on a wafer are ordinarily wafer numbers, whereas the marks recorded on a wafer case are ordinarily one-dimensional or two-dimensional codes. An array of bar patterns with black and white colors, which is a so called “bar code”, is used as one-dimensional code. A stacked type of one-dimensional bar codes piled up and a matrix type of an array shaped like a grid are used.

A wafer case is typically made of a polymer material such as a polycarbonate (PC). A mark on a wafer case may be formed using the following methods:

(1) Attaching a sheet or substrate, on which a code is marked, to a wafer case.

(2) Marking a code directly on a wafer case.

However, these methods have the following problems:

In method (1), the sheet or substrate attached to the wafer case may come away during a wafer production process, and the wafers in the wafer case may be polluted by an adhesive agent that is used in the attachment of the sheet to the wafer case.

Method (2), in which a code is marked directly on a wafer case, has the following problems:

(i) When the wafer case is transparent or the part where the code is marked is transparent, most of the laser light for forming the mark passes through the wafer case, and thus the code cannot be certainly marked on the wafer case.

(ii) As a solution of (i), the wafer case material may be carbonated by mixing a heating element into the material and then reacting and heating the heating element with a laser light. For example, U.S. Pat. No. 5,445,271 discloses a wafer case containing 0.0001 to 0.5 percent by weight of Si, Ca, Ba, Na, K, C, Ti, Al or Mg as the heating element. However, there is a problem that some wafers in the wafer case are polluted by these elements. There is also a problem that marking can not be sufficiently fulfilled by insufficient carbonization of the wafer case material that is caused by unequal heating of the heated part.

(iii) Since laser light heats the entire area in a thickness direction of the wafer case, the portion that is irradiated by the laser light is degraded. Thus, the wafer case cannot fulfill its essential role.

### SUMMARY OF THE INVENTION

Laser light having a wavelength out of a wavelength region of visible light is irradiated on a wafer case that is made of a polymer having a low transmittance of laser light and a high transmittance of visible light. The irradiated portion of the wafer case material foams, blackens, melts or evaporates. Thus, convexities and concavities are formed on the wafer case and the irradiated portion gets discolored, and wafer case information can thereby be recorded thereon. For example, the wafer case information may be recorded as one-dimensional or two-dimensional codes. Further, two or more laser lights may be irradiated on the same area of the wafer case surface from different directions to the wafer case surface. Thus, since only the area close to the surface that is irradiated by the laser lights foams, blackens, melts or evaporates, wafer case information can be recorded there.

### BRIEF DESCRIPTION OF THE DRAWINGS

FIG. 1 is a perspective view of a wafer case having identification marks in accordance with the present invention.

FIG. 2 is a graph showing a relation between transmittance and wavelength of light in a polycarbonate.

FIG. 3 is a view showing a concavity and a convexity formed by laser light.

FIG. 4 is a view showing a path of a laser light entering a wafer case.

FIG. 5 is a view showing paths of laser lights entering a wafer case from two different directions.

FIG. 6 is a view showing an example of two dimensional code having a dot pattern in accordance with the present invention.

FIG. 7 is a perspective view of another wafer case having identification marks in accordance with the present invention.

FIG. 8 is a view showing another embodiment using three laser lights in accordance with the present invention.

### DETAILED DESCRIPTION OF THE INVENTION

A plastic container for thin substrates holds or keeps, for example, thin semiconductor substrates such as silicon substrates and compound semiconductor substrates; reticles; photomask substrates; and discs such as magnetic disks and compact disks.

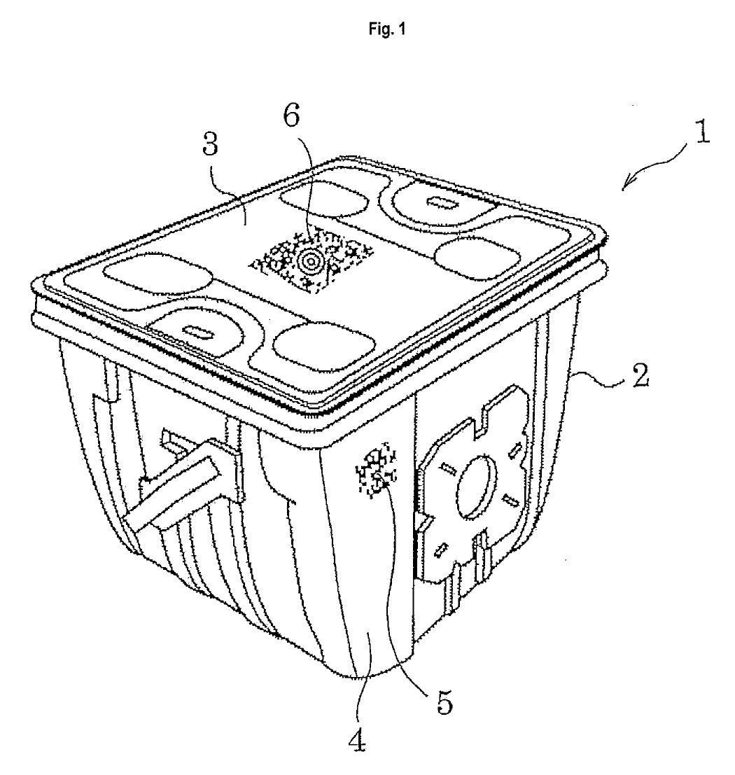

FIG. 1 shows a plastic container for thin substrates (hereinafter called a “wafer case” or “wafer carrier box”) that is used for holding or keeping a semiconductor substrate (hereinafter called “wafer”) such as a silicon substrate and a compound semiconductor substrate. The wafer case 1 is opened at one side and consist primarily of a housing 2 containing a wafer carrier (not shown), which directly supports or holds wafers, and a lid member 3 covering the opening of the housing 2. Though the housing 2 and the lid member 3 are combined in FIG. 1, they can be separated. The housing 2 separates from the lid member 3 when the wafer carrier is taken in and out of the housing 2.

On a side wall 4 of the housing 2 of the wafer case 1, a one-dimensional or two-dimensional code 5 is marked as an identification sign for processing histories and controls of the wafers kept in the wafer case. The code 5 also records some information for transportation and keeping of the wafer case 1, in addition to the information above. This various information is referred to herein as “wafer case information”. The code 5 may be marked under a bottom part (or on a bottom face) of the housing 2 of the wafer case 1. Further, a code (one-dimensional or two-dimensional) 6 may be marked on an upper face of the lid member 3 of the wafer case 1. The code 6 records the wafer case information. The codes 5 and 6 are read out by a code reader placed on the way of wafer processing. The wafer case and the wafers in it are handled, carried, kept, etc., in the following process in accordance with the information of the codes 5 and 6.

As a method for marking wafer case information on the wafer case, laser light can be irradiated on the wafer case after it is arranged on a place where laser light can be irradiated. When laser light is irradiated on the wafer case, the portion of the wafer case irradiated by the laser light absorbs energy of the laser light and generates heat. The heat generation forms convexities and concavities in the wafer case material by foaming, upheaving, melting or evaporating. Or it causes the wafer case material to blacken or change color.

Laser light passes through a transparent wafer case and thus cannot sufficiently heat the irradiated portion of the wafer case. However, even though the wafer case is transparent to visible light, this invention provides methods to mark a very brilliant code on it using laser light.

When a wafer case is made of a polymer material transparent to visible light, the transparent polymer material is not transparent in wavelength regions of all lights (or electromagnetic rays). Some polymer materials have a low transmittance in ultraviolet or X-ray wavelength regions that have shorter wavelengths than the wavelengths of visible light (conversely, the light absorptance is higher). Some polymer materials have a low transmittance in infrared wavelength regions that have longer wavelengths than the wavelengths of visible light (conversely, the light absorptance is higher).

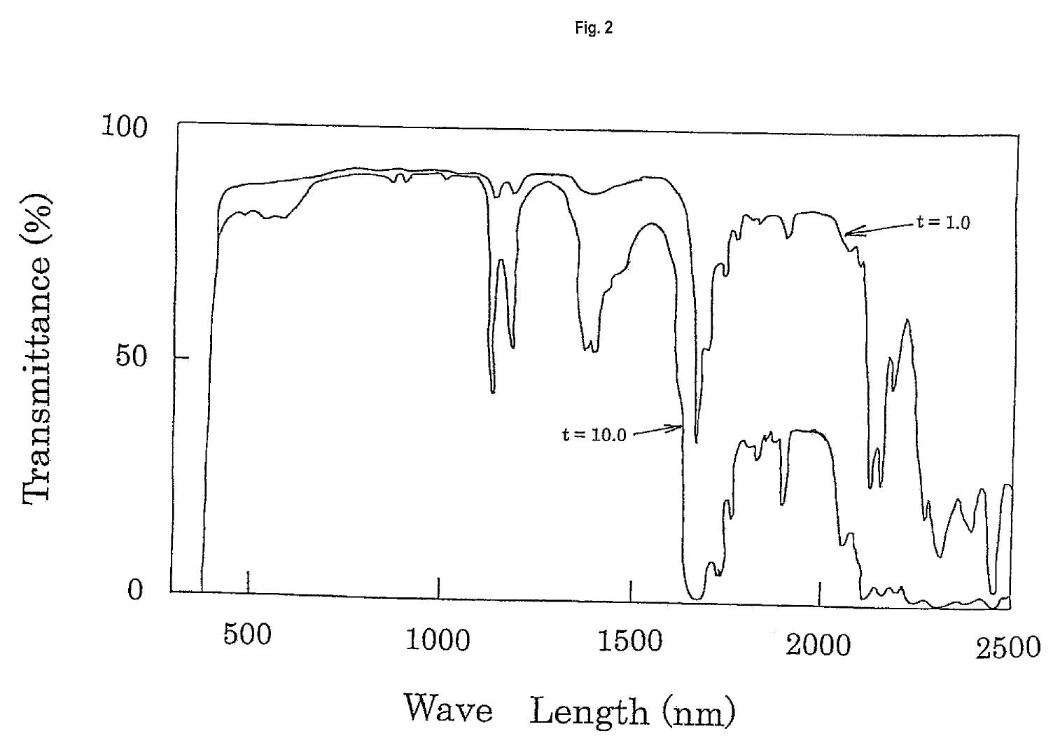

FIG. 2 shows a relation between the wavelength and transmittance of light for polycarbonates that are 1.0 mm (t=1.0) and 10.0 mm (t=10.0) in thickness. The transmittance of the polycarbonate is equal to or greater than 80% in the wavelength region of the visible light, whereas it is equal to or less than 10% (almost 0%) in the ultraviolet region that is equal to or less than 350 nm in wavelength. Also, it is low in the infrared region that is 1100 nm-1200 nm, near 1400 nm (1350 nm-1450 nm), 1500 nm-1800 nm, and more than 1200 nm. When, on a wafer case made of a polymer material that has a low transmittance in the ultraviolet region (including a near-ultraviolet region) or in the infrared region (including a near-infrared region), laser lights that have their wavelengths are irradiated, and the irradiated portion of the wafer case generates heat. Thus, concavities and convexities are formed by foaming, swelling, melting, or evaporating in the irradiated portion. Or the wafer case materials of the irradiated portion are blackened or colored.

The term “transparent material” to visible light means a material that has an average transmittance equal to or more than about 70%, preferably about 80%, and more preferably about 85%. Also, a low transmittance of light having a wavelength other than that of visible light is equal to or less than about 30%, preferably about 20%, more preferably about 10%, and most preferably about 1%. Further, a high transmittance of light means a transmittance that is equal to or more than about 70%, preferably about 80%, more preferably about 85%, and a low transmittance of a light means a transmittance that is equal to or less than about 30%, preferably about 20%, more preferably about 10%, and most preferably about 1%.

A laser light that has an ultraviolet wavelength equal to or less than 300 nm can be used in a wafer case that is made of the polycarbonate described above. For example, an excimer laser, a diode-pumped laser or a lamp pumping laser can be used. ArF (193 nm), KrF (248 nm), or XeCl (308 nm), for example, can be used as an excimer laser. Also, a polycarbonate that has a low transmittance in the infrared wavelength region can be used. Accordingly, for example, a code is marked on a wafer case made of a polycarbonate using an infrared laser such as a glass laser, a semiconductor laser, or a carbon dioxide gas laser.

The portion of the wafer case irradiated by a laser light generates heat, and is carbonized and blackened by absorbing the energy of the laser light. In this case, a one-dimensional or a two-dimensional code can be prepared on the irradiated part by regularly forming the portion that the laser light irradiates and the portion that the laser light does not irradiate. Or, the concavities and convexities can be formed by making the wafer case material foam, melt and evaporate by the heat of the laser light. Since the portion where concavities and convexities are formed by the laser light diffusely reflects illuminated light, they are identified as black or white by a code reader and have representational function.

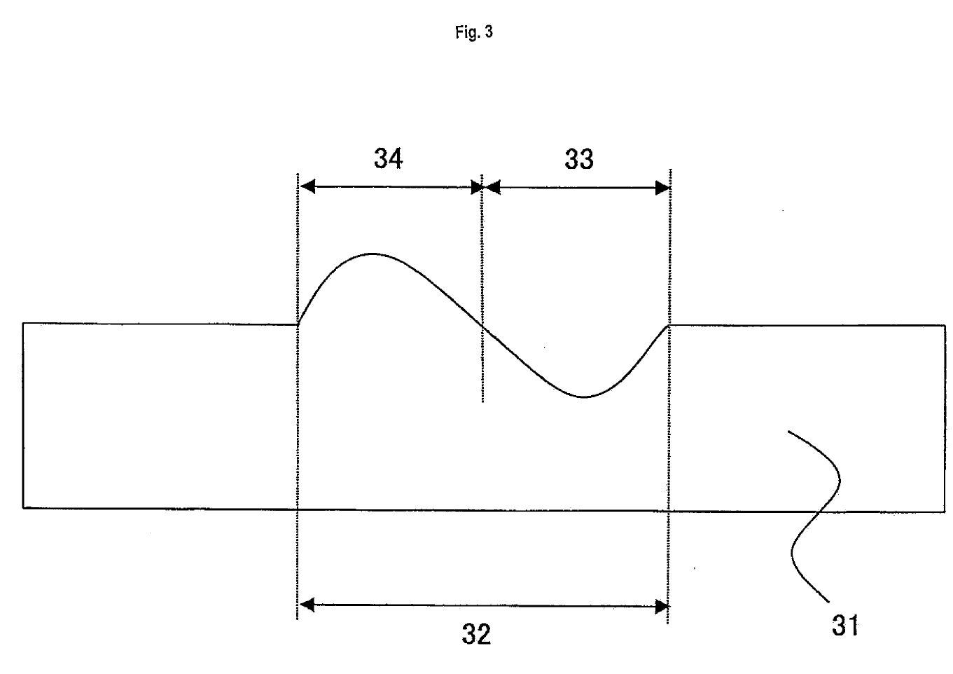

FIG. 3 schematically illustrates concavities and convexities formed by a laser light. In FIG. 3, a laser light is irradiated on the specific area 32 of a wafer case 31, and thus the portion where the laser light is irradiated absorbs more than a constant amount of energy and melts. After the portion is cooled, a part of the melted portion hollows (that is, forms a concave part 33) and a part of the melted portion swells (that is, forms a convex part 34). Thus, a concavity and a convexity are formed in the portion irradiated by the laser light. When a one-dimensional code, that is to say a bar code, is formed, the laser light is irradiated linearly and repeatedly.

When a two dimensional code is formed, for example, when a two dimensional pattern is linearly formed, the laser light is irradiated linearly as when a one dimensional code is formed, and is scanned two-dimensionally. When a two dimensional code such as a polka-dot pattern is formed, the laser light is repeatedly irradiated in a rectangular or circle shape. Thus, a desired code can be formed in a given area on the wafer case.

When a wafer case material is evaporated by a laser light, the portion where the laser light is irradiated mainly hollows (that is, it becomes concave). When a wafer case material is foamed and upthrusted by a laser light, the portion where the laser light is irradiated mainly swells (that is, it becomes convex). Further, the portion of the wafer case where the laser light is irradiated can be blackened or ebonized. In such cases, a desired code can be formed in the given area on the wafer case as discussed previously.

A transparent wafer case is not perfectly transparent. That is, the transmittance of light is not 100%. Accordingly, when a light for reading a code is irradiated from above the code part of the wafer case, code information can be read out utilizing the difference of the light intensity between reflected light from a flat plane, which has no black parts or concavities and convexities, and reflected light from an irregular plane, which has black parts or concavities and convexities.

When a laser light is irradiated into a wafer case, in general, the whole portion in the thickness direction of the wafer case in the irradiated part is blackened, or concavities and convexities are formed there. That is, to foam, melt, evaporate or blacken a wafer case using only one laser light, a laser light having very large energy needs to be irradiated. While the irradiated laser light passes through the wafer case, the energy of the laser light is absorbed by the wafer case material. The absorbed energy changes into heat and thus makes the wafer case foam, melt, evaporate, or blacken. Hereinafter, this energy is called energy for heating a wafer case. This may have an adverse effect on the wafers inside the wafer case. That is, the portion where the laser light is irradiated in the wafer case may be degraded from the surface to the rear surface of the wafer case. By the degradation, the strength of the wafer case may become smaller, or the wafers inside the wafer case may be polluted.

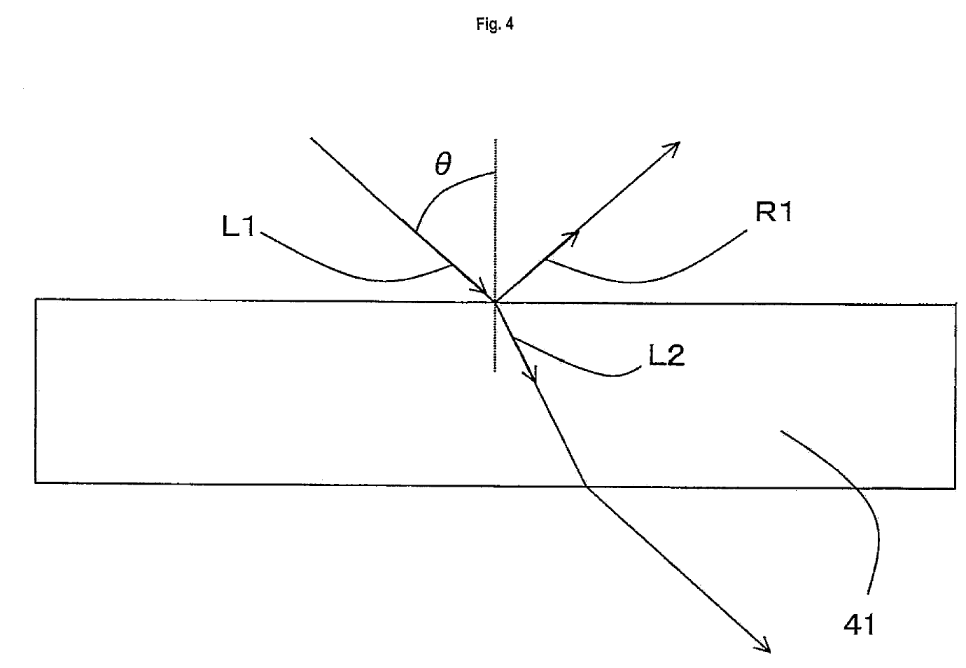

Also, when a laser light is irradiated from a direction oblique to a surface of a wafer case, most of the laser light is irradiated within the wafer case (which is an incident light) and some of the laser light is reflected. Some energy of the incident light is absorbed into the wafer case material and thus is converted into heat. As illustrated in FIG. 4, a laser light L1 having an intensity E1 enters a wafer case at an angle of θ from a direction oblique to a direction vertical to a surface of a wafer case 51. Some of the laser light goes into the wafer case (an incident light L2 having an intensity E2), while some of it reflects (a reflected light R1 having an intensity Er). Some energy of the laser light is converted to heat (the energy Eh), and thus the wafer case material is blackened, or concavities and convexities are formed there. A minimum energy for blackening the wafer case or forming a concavity and a convexity is defined as Ee. When Eh is greater than Ee, the portion of the wafer case where the laser light is irradiated blackens, or the concavities and the convexities are formed there. However, since it is very difficult to accurately find a value of Ee and to control Eh, the laser light having the intensity E1 needs to be irradiated by considering Eh having a much greater energy than Ee. As a result, since heat having a greater energy than Ee is supplied in the whole of the thickness direction of the wafer case, it is difficult to make only the surface neighborhood of the wafer case blacken and to form concavities and convexities on only the surface neighborhood of the wafer case. Instead, the whole area from the surface to the rear surface of the wafer case is blackened, and concavities and convexities are formed in the whole area. This phenomenon becomes more remarkable as the transmittance of the wafer case becomes larger.

Therefore, this invention is characterized by irradiating a plurality of laser lights having Eh less than Ee from at least two directions as the method for blackening only the surface neighborhood of the wafer case or forming concavities and convexities in only the surface neighborhood of the wafer case without damaging the inside of the wafer case. Though these laser lights are irradiated so that their incident directions are different, the points where they are irradiated are corresponding on the wafer case. Thus, it is possible to blacken only the surface neighborhood of the wafer case or to form the concavities and the convexities on only the surface neighborhood of the wafer case.

In this specification, a surface neighborhood of a wafer case indicates the portion equal to or less than about a half thickness of the wafer case. For example, when a thickness of a wafer case is 1 mm, the surface neighborhood of the wafer case is the portion equal to or less than about 0.5 mm in the thickness of the wafer case. When a thickness of a wafer case is 5 mm, the surface neighborhood of the wafer case is the portion equal to or less than about 2.5 mm in the thickness of the wafer case. However, when the portion where the concavities and the convexities are formed or the carbonization is carried out does not reach the rear surface of the wafer case even over the half thickness of the wafer cases the surface neighborhood of the wafer case indicates the portion equal to or more than a half thickness of the wafer case.

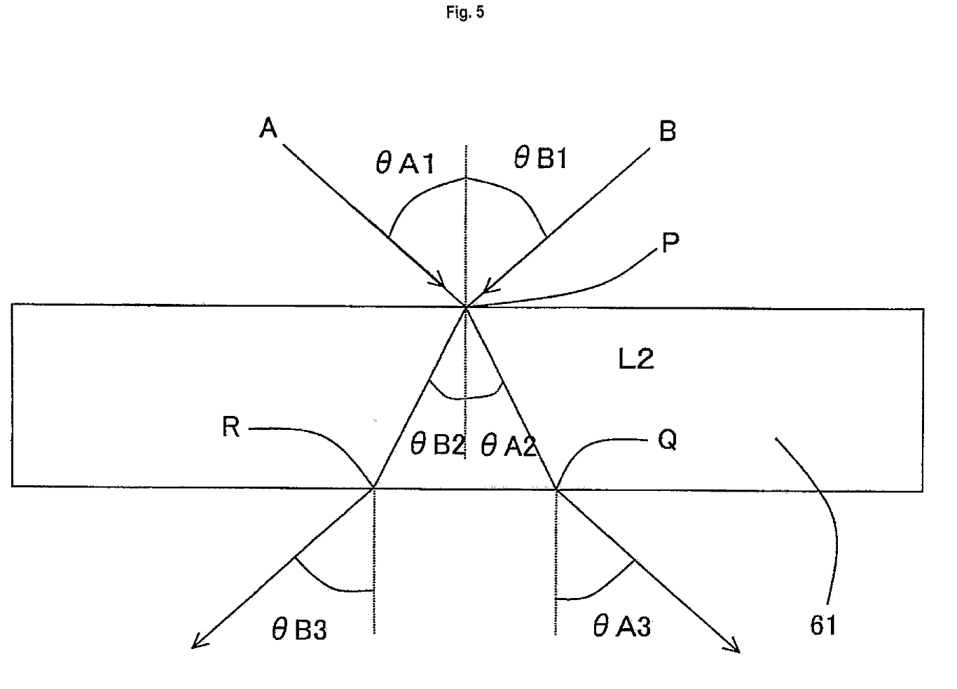

This is illustrated in FIG. 5. Laser lights A and B are irradiated on a surface point P of a wafer case 61 from two different directions. The laser light A is irradiated on the wafer case 61 at an angle of θ A1 to the vertical direction in the surface P of the wafer case 61. The intensity of the laser light A irradiated on the wafer case 61 is EA1. The laser light A goes at an angle of θ A2 to the vertical direction in the surface P of the wafer case 61 after being irradiated on the wafer case 61. The intensity of the laser light A shortly after being irradiated on the wafer case 61 is EA2. The laser light A then exits the wafer case 61 at an angle of θ A3 to the vertical direction at a rear surface point Q of the wafer case 61. When the mediums are same outside of the surface and outside of the rear surface of the wafer case, θ A1 equals θ A3. The intensity of the laser light A just before it exits the wafer case 61 at a rear surface point Q of the wafer case 61 is EA3. The intensity of the laser light A shortly after it exits the wafer case 61 at the rear surface point Q of the wafer case 61 is EA4. Since some of the laser light A scatters and reflects on the surface P of the wafer case and some of the laser light A is absorbed in the surface P of the wafer case, EA2 is smaller than EA1. Some energy of the laser light A absorbed in the surface of the wafer case 61 changes to heat and thus heats the wafer case 61. The energy of the laser light A that is absorbed on the surface of the wafer case 61 and heats the wafer case 61 is EAH. Laser light is also scattered and absorbed within the wafer case 61. Accordingly EA3 is smaller than EA2. Since some of the laser light A is scattered and reflected at the rear surface point Q of the wafer case 61 and some of the laser light A is absorbed at the rear surface point Q of the wafer case 61, EA4 is smaller than EA3. Some of the energy of the laser light A absorbed inside of the wafer case changes to heat and thus heats the wafer case 61. In this manner, though the wafer case 61 is heated by the laser light A, if the temperature of the heated portion does not exceed a foaming temperature, a melting point, a boiling point or a blackening temperature of the wafer case material, the portion where the laser light is irradiated in the wafer case 61 does not foam, melt, evaporate or blacken. At the surface point P of the wafer case, if EAH is smaller than the energy to foam, melt, evaporate or blacken the wafer case material, the wafer case material does not foam, melt, evaporate or blacken at the point P.

Likewise, the laser light B is irradiated on the wafer case 61 at an angle of θ B1 to the vertical direction in the surface P of the wafer case 61. The intensity of the laser light B irradiated on the wafer case 61 is EB1. The laser light B goes at an angle of θ B2 to the vertical direction in the surface P of the wafer case 61 after being irradiated on the wafer case 61. The intensity of the laser light B shortly after being irradiated on the wafer case 61 is EB2. The laser light B then exits the wafer case 61 at an angle of θ B3 to the vertical direction at a rear surface point Q of the wafer case 61. When the mediums are same between outside of the surface and outside of the rear surface of the wafer case, θ B1 equals to θ B3. The intensity of the laser light B just before it exits the wafer case 61 at a rear surface point Q of the wafer case 61 is EB3. The intensity of the laser light B shortly after it exits the wafer case 61 at the rear surface point Q of the wafer case 61 is EB4. Since some of the laser light B scatters and reflects on the surface P of the wafer case and some of the laser light B is absorbed in the surface P of the wafer case, EB2 is smaller than EB1. Some energy of the laser light B absorbed in the surface of the wafer case 61 changes to heat and thus heats the wafer case 61. The energy of the laser light B that is absorbed on the surface of the wafer case 61 and heats the wafer case 61 is EBH. Laser light is also scattered and absorbed within the wafer case 61. Accordingly, EB3 is smaller than EB2. Since some of the laser light B is scattered and reflected at the rear surface point Q of the wafer case 61 and some of the laser light B is absorbed at the rear surface point Q of the wafer case 61, EB4 is smaller than EB3. Some of the energy of the laser light B absorbed inside of the wafer case changes to heat and thus heats the wafer case 61. In this manner, though the wafer case 61 is heated by the laser light B, if the temperature of the heated portion does not exceed a foaming temperature, a melting point, a boiling point or a blackening temperature of the wafer case material, the portion where the laser light is irradiated in the wafer case 61 does not foam, melt, evaporate or blacken. At the surface point P of the wafer case, if EBH is smaller than the energy to foam, melt, evaporate or blacken the wafer case material, the wafer case material does not foam, melt, evaporate or blacken at the point P.

Even if the laser light A or B does not independently have the energy that enables the laser light A or B to make the wafer case material foam, melt, evaporate or blacken, the energy of the light A or B can be respectively selected so that the wafer case material is foamed, melted, evaporated or blackened by the combined energies of the lights A and B. For example, when the energy to foam a wafer case is EK, if EAH plus EBH is larger than EK even though EAH or ABH is smaller than EK, the wafer case material foams at the surface point P of the wafer case. When the energy to melt a wafer case is EM, if EAH plus EBH is larger than EM even though EAH or ABH is smaller than EM, the wafer case material melts at the surface point P of the wafer case. When the energy to evaporate a wafer case is ES, if EAH plus EBH is larger than ES even though EAR or ABH is smaller than ES, the wafer case material evaporates at the surface point P of the wafer case. When the energy to blacken a wafer case is ET, if EAH plus EBH is larger than ET even though EAH or ABH is smaller than ET, the wafer case material blackens at the surface point P of the wafer case.

Further, since the light paths of the laser lights A and B are different after entering the wafer case 61, the laser lights A and B do not overlap at any place other than the wafer case surface P (or the neighborhood of the surface P). Accordingly, when the energy by which the laser light A entering the wafer case heats the wafer case is EAH2, and when the energy by which the laser light B entering the wafer case heats the wafer case is EBH2, if EAH2 or EBH2 is smaller than EK, the wafer case material does not foam. If EAH2 or EBH2 is smaller than EM, the wafer case material does not melt. If EAH2 or EBH2 is smaller than ES, the wafer case material does not evaporate. If EAH2 or EBH2 is smaller than ET, the wafer case material does not blacken.

As described above, only one point of the wafer case surface point or only the neighborhood of the point of the wafer case surface can be foamed, melted, evaporated or blackened by irradiating the laser lights from two different directions on the wafer case. Code, etc. can thereby be marked on a minimal point of the wafer case surface.

Further, by irradiating laser lights from three or more different directions on the wafer case surface, code, etc. can be marked on a minimal point or a neighborhood containing the minimal point of the wafer case surface. When laser lights are irradiated from three or more different directions on the wafer case surface, since a laser light having a smaller intensity can be used as compared with a case in which laser lights are irradiated on the wafer case surface from two directions, it is further possible to reduce the damage that the laser lights cause to areas other than the minimal point on the wafer case. In this case, since a laser light with a smaller power can be used, the cost of the laser equipment can also be reduced.

According to the invention, it is also possible to use a laser light having a wavelength in the range of visible light in materials transparent to visible light. That is, some laser light is inevitably absorbed even in materials transparent to visible light. For example, as shown in FIG. 2, the average absorption of a polycarbonate is about 10% in the range of visible light. A laser light having a large amount of energy needs to be used to melt a wafer case material having a wavelength within the range of visible light. Accordingly, when the wafer case material is melted by only one laser light, it is very difficult to melt only the surface portion of the wafer case. That is, even though it is desired to melt only the wafer case surface by one laser light, the rear surface of the wafer case will also be melted and thus a damage is caused from the surface to the rear surface of the wafer case. Since this results in reduced strength and pollution of the wafer case, the wafer case cannot fulfill its role.

This invention is applicable to such a case. That is, laser lights having energies of an extent that does not melt a wafer case material are irradiated from at least two different directions to a surface of the wafer case. Two or more laser lights are irradiated on the wafer case so that they shine on the same portion of the wafer case surface.

After the lasers enter the wafer case, they go in different directions, respectively. As the laser lights are visible lights, some of their energies are absorbed into the wafer case material, whereas most of their energies pass the wafer case. A specified area of the wafer case surface on which a plurality of laser lights collect, is heated and thus is foamed, melted, evaporated or blackened, whereas the other area of the wafer case where the laser lights pass is heated, but is not foamed, melted, evaporated or blackened. If the wafer case material is not foamed, melted, evaporated or blackened by collecting two laser lights because their energies are small, further other laser lights are irradiated from other directions on the same portion of the wafer case surface. By this means, only the wafer case surface is formed, melted, evaporated or blackened, and thus data can be recorded.

An angle (θ A1 or θ B1, etc.) at which a laser light is irradiated to the wafer case must be changed by a refractive index of the wafer case material, or by the thickness of the wafer case. It must be also changed by the distance from the wafer case surface where the area within the wafer case is to be foamed, melted, evaporated or blackened, or it must be changed by the number of laser lights or their energies. However, since a plurality of laser lights influence each other when their angles are too small, their angles need to be more than some degree. At one point (for example, point P in FIG. 5) on the wafer case surface or at the place away from the neighborhood of the point, the sum of the energies of a plurality of laser lights heating the wafer case must not surpass a forming energy, a melting energy, an evaporating energy or a blackening energy of the wafer case. The incident angle of the laser light to the vertical direction of the wafer case surface is preferably about 70 degrees or less, more preferably about 60 degrees or less, more preferably about 50 degrees or less, more preferably about 40 degrees or less, and more preferably about 30 degrees or less. However, according to the invention, some laser lights can be irradiated from an angle greater than these angles. It is important that in the area where the wafer case material is not to be foamed, melted, evaporated or blackened, the sum of the energies of a plurality of laser lights does not surpass the forming energy, melting energy, evaporating energy or blackening energy of the wafer case.

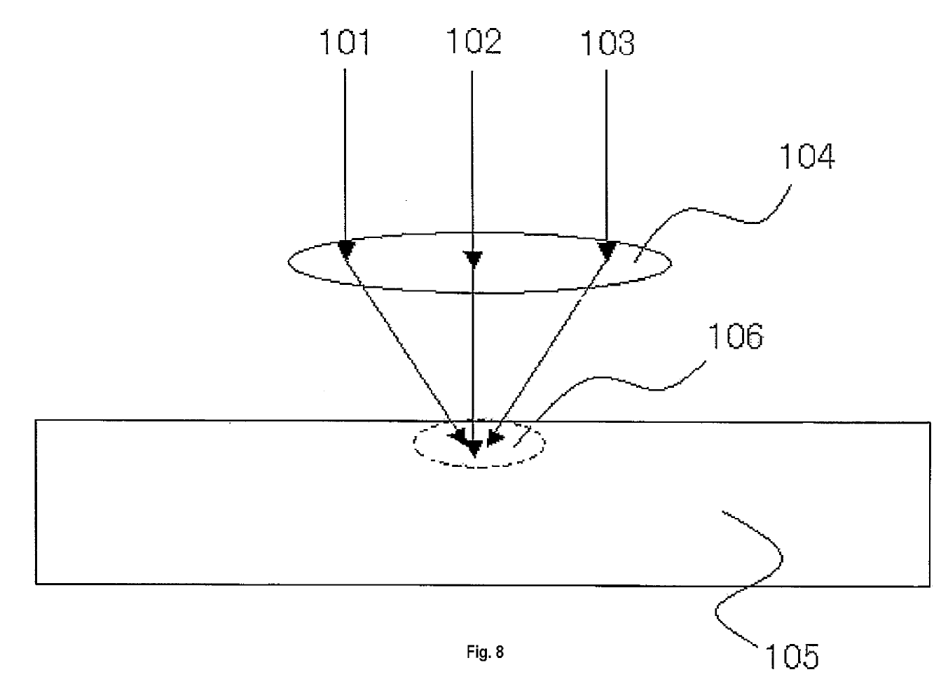

FIG. 8 is a view showing another embodiment of the present invention. Three laser lights 101, 102 and 103 vertically enter a lens 104 and are focused on a surface neighborhood 106 of a wafer case 105. That is, the three laser lights vertically enter the lens 104 by adjusting a location of the lens 104 so that a focus of the lens 104 is connected on the surface neighborhood 106 of a wafer case 105. Even though one laser light does not have an energy to make the wafer case 105 foam, melt, evaporate, or blacken, the wafer case 105 can be foamed, melted, evaporated, or blackened if the combined energy of the three laser lights 101, 102 and 103 is larger than the energy needed to make the wafer case 105 foam, melt, evaporate or blacken.

Though three laser lights are used in the embodiment shown in FIG. 8, the wafer case information can be written in the wafer case using two laser lights by adjusting the individual energies of the laser lights so that their combined energy makes the wafer case foam, melt, evaporate, or blacken. In addition, wafer case information can be written in the wafer case using four or more laser lights. It can also be written in the wafer case using different kinds of laser lights. In addition to being led directly to the lens, the laser light can be led to the lens through an optical fiber, a light guide, a mirror, or other lens, etc.

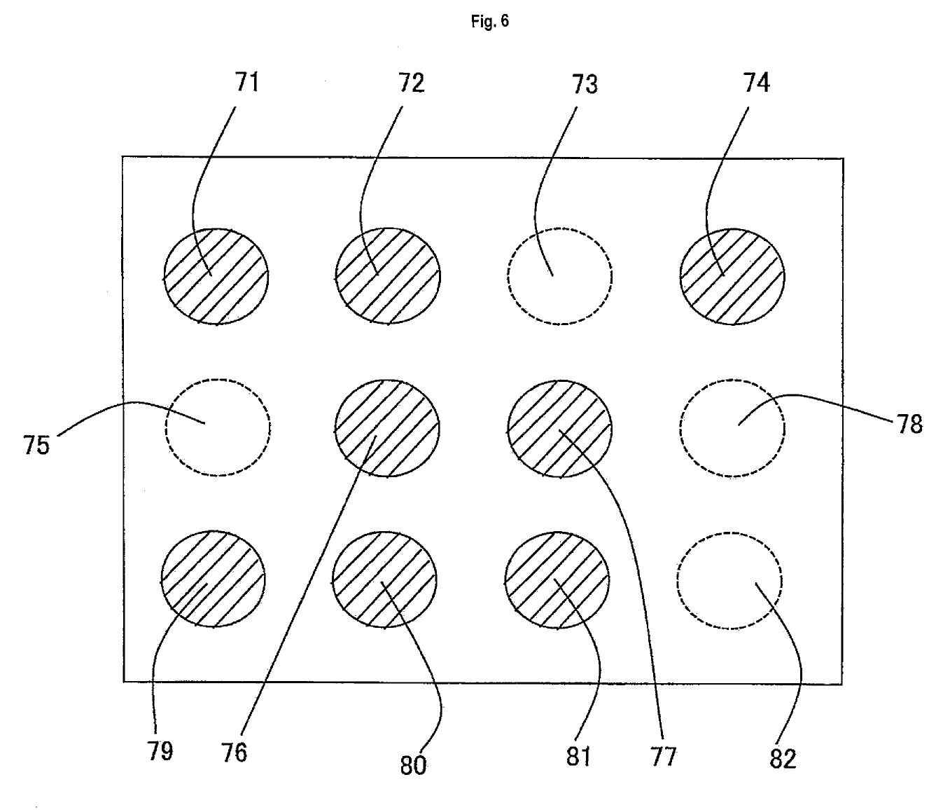

FIG. 6 shows an example of a two dimensional code formed in a dot pattern using this invention. In the area shown in FIG. 6, a twelve dot pattern is marked. For example, a laser light is irradiated on a circle area such as 71. Thus, a concavity portion and a convexity portion, or blackening portions are formed. As a laser light is not irradiated on the circle area indicated with dash lines such as 73, the area is flat. A laser light for reading out codes is irradiated from above the area having such dot pattern. Thus, information can be read out using the difference of the strength between the reflected light from the concavity and convexity portions or the blackening portion 71 and the reflected light from the flat portion 73, for example, by identifying the portion of the strong reflected light as a signal of “1” and the portion of the weak reflected light as a signal of “0.” Conversely, information can be written since the portion where the laser light is irradiated and the portion where the laser light is not irradiated can be identified. In FIG. 6, the shaded area of a circle surrounded by a real line is the portion where light is irradiated, whereas the blank area of a circle surrounded by a dash line is the portion where light is not irradiated. That is, light is irradiated at portions 71, 72, 74, 76, 77, 79, 80 and 81, and is not irradiated at portions 73, 75, 78 and 82. The laser light is also not irradiated in areas outside of the circle area. When the strength of the reflected light from portions where the laser light is irradiated is relatively smaller than the strength of the reflected light from portions where the laser light is not irradiated, by reading out only the circles surrounded by a real line, the twelve digit binary number of “001010010001” can be obtained from the area having twelve circles. That is, from the area having these twelve circles, 2 raised to the power 12, that is, 4096 different data can be obtained. In this way, wafer case information can be written on a wafer case surface or a neighborhood of a wafer case, and can be read out.

Wafer case information can be written in the wafer case by scanning a laser light. Also, wafer case information can be written in the wafer case by irradiating a laser light through a mask in which the information is pre-written.

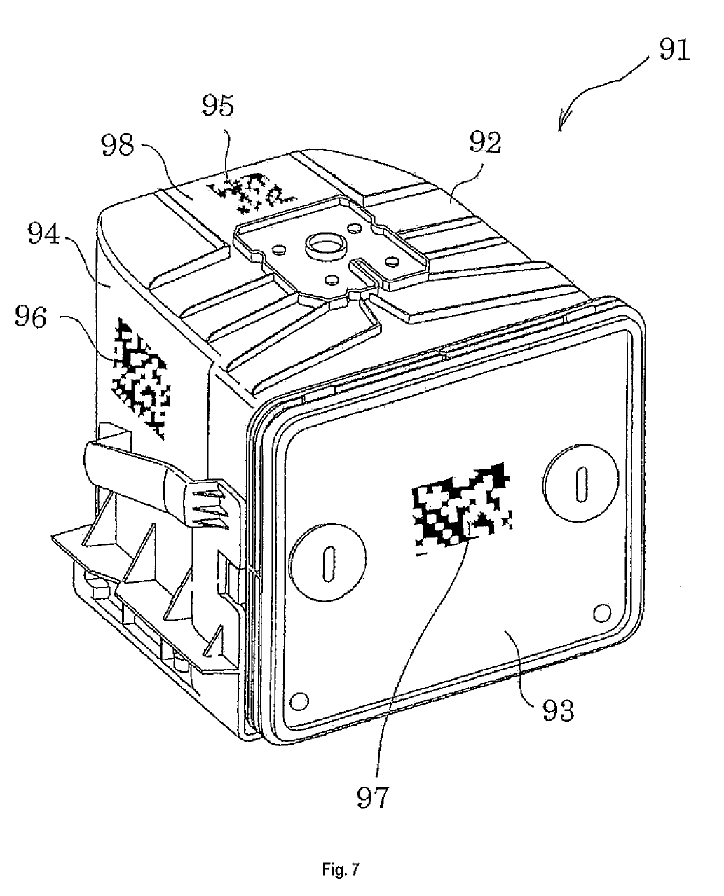

FIG. 7 shows a wafer case having a shape different from that of the wafer case of FIG. 1, which has wafer case information in accordance with this invention. The wafer case 91 is formed into a basket shape having an opening at its one side, and contains a housing 92, which stores a wafer carrier (not shown) directly supporting wafers, and a lid member 93 covering the opening of the housing 92. Though the lid member 93 is shown as joined together with the housing 92 in FIG. 7, the lid member 93 can be separated from the housing 92. In general, when the wafer carrier is taken in and out of the wafer case, the lid member 93 is separated from the housing 92.

Codes 95 and 96, which are shown as two-dimensional codes in FIG. 7, are marked as wafer case information on the lateral sides 94 and 98 of the housing 92 of the wafer case 91. Further, a code 97, which is also shown as two-dimensional code in FIG. 7, is marked on the upper plane of the lid member 93 of the wafer case 91. The code 97 has wafer case information. These codes 95, 96 and 97 are read out by a code reader set up on the way of the wafer process and the subsequent processing, transferring, storing, etc. of the wafer, etc. are done according to the information of the codes 95, 96 and 97.

The above description assumes that when a transparent polymer material is irradiated by a laser light and melted, etc., the polymer material after solidification is also transparent. However, a transparent polymer material is a so-called amorphous material in which the crystallinity is generally small. When such amorphous materials are irradiated by a laser light and melted, etc., they may be finely crystallized or poly crystallized after their solidification. Such a crystalline state makes the transparency of the polymer material lower and makes the absorbance of light larger. When a light for reading out a code is irradiated on such portions, a ratio of diffused reflection on the portions becomes larger (since fine concavities and convexities based on the crystalline characteristics may be formed). Also, without a laser light putting into the wafer case passing it, the ratio of light absorbed into the wafer case material increases. Accordingly, in such a case, even though light for reading out a code is irradiated from above, since there are almost no reflected lights which return to the original position, it is possible to write information there.

This invention enables a laser light having an output power that is not too large to be irradiated into a transparent wafer case from different directions. Accordingly, since it is not necessary to put heating elements, such as Si, Ca, Ba, Na, K, C, Ti, Al, or Mg, etc., into the wafer case material, the problem of pollution of wafers that are kept in the wafer case by such heating elements does not occur. When there is not a problem even if wafers are polluted by the heating elements, or when the problem that these heating elements pollute wafers which are kept in the wafer case does not occur, there is no problem in this invention even if these heating elements are contained in the wafer case.

A polypropylene, a cyclic olefin, a polyethylene, a polyethylene terephthalate, a polystyrene, a polyester, an acrylic, etc., can be used in addition to a polycarbonate as a wafer case material that is transparent in the optical wavelength band. This invention can be used in a wafer case made of these polymers. For example, though the average transparency of a polyethylene (a type of electron beam irradiation) in the optical wavelength band is 70% or more (where the thickness is 0.04 mm), its transparency in the ultraviolet light wavelength band is 40% or less (where the thickness is 0.04 mm). Accordingly, for example, this invention can be applied using a laser light having a wavelength less than 250 nm in a wafer case made of polyethylene. Though the average transparency of a polystyrene in the optical wavelength band is 80% or more (where the thickness is 0.03 mm), its transparency in the ultraviolet light wavelength band less than 250 nm is 10% or less (where the thickness is 0.03 mm). Accordingly, for example, this invention can be applied using a laser light having a wavelength less than 250 nm in a wafer case made of the polystyrene. Though an average transparency of a polyester in the optical wavelength band is 70% or more (where the thickness is 0.04 mm), its transparency in the ultraviolet light wavelength band less than 300 nm is 10% or less (where the thickness is 0.04 mm). Accordingly, for example, this invention can be applied using a laser light having a wavelength less than 300 nm in a wafer case made of polyester.

As discussed previously, this invention can use almost all laser lights in all wavelength bands. For example, the invention can use a gas laser such as an He—Ne (helium-neon) laser, an Ar (argon) laser, a carbon dioxide gas laser, an excimer laser, or a nitrogen laser; a solid state laser such as a ruby laser, a titanium-sapphire laser, a YAG laser, a glass laser, a Nd (neodymium) laser, a solid state green laser, or a fiber laser; a metal vapor laser such as an He—Cd (helium-cadmium) laser, a copper vapor laser, or a gold vapor laser; a semiconductor laser; a liquid laser such as a dye laser; a chemical laser such as an HF (hydrogen fluoride) laser; or a free electron laser.

This invention has an advantage in that it can use a laser light having a wavelength in the visible light range in a wafer case transparent to visible light, as discussed previously. For example, the invention can use a gas laser such as an He—Ne (helium-neon) laser or an Ar (argon) laser; a solid state laser such as a ruby laser or a titanium-sapphire laser; a metal vapor laser such as an He—Cd (helium-cadmium) laser, a copper vapor laser, or a gold vapor laser; a semiconductor laser; or a liquid laser such as a dye laser. The laser lights in the visible wavelength have an advantage in that a human can directly see them.

An abrasion process can be used when an excimer laser light is employed in this invention. That is, the excimer laser light is irradiated on a desired portion of the wafer case, thus the intermolecular chemical bonds are cut, and the gasified materials are scattered to the outside such as the atmosphere. Also, a mask in which wafer case information is pre-written can be used in the abrasion process using an excimer laser light. In this way, wafer case information can be written by forming concavities and convexities, etc. in the given portion of the wafer case.

Further, as discussed above, this invention can use a laser having a small output power. That is, it is important to collect laser lights on one point of a wafer vase from multiple directions. This invention has another advantage that damage to the wafer case is minimized because it is not necessary to irradiate laser lights that have excessive energy on the wafer case. For example, though an argon ion laser can develop from 100 mW to 20 W of output power, multiple laser lights having small output powers such as about 100 mW can be irradiated on the wafer case in this invention.

It goes without saying that the method of this invention for irradiating laser lights from multiple directions can be also used in a wafer case that is not transparent to visible light.

Though this invention has been explained mainly for a wafer case, it can also be used in a resin-made basket that keeps thin sheets or substrate (a wafer case is a kind of a resin-made basket). Further, this invention can also be used in general resin products.

1. A method for writing wafer case information on a given area of a wafer case by irradiating a laser light, wherein said wafer case is made of a polymer material which has a high transmittance of light in a visible wavelength region and a low transmittance of light in a wavelength band X out of the visible wavelength region.

2. The method of claim 1, wherein an average transmittance of light of a material of the wafer case is 80% or more in the visible wavelength region, and 30% or less in the wavelength band X.

3. The method of claim 2, wherein the polymer material is a polycarbonate

4. The method of claim 3, wherein the wavelength band X is a wavelength region of 50 nm-100 nm.

5. The method of claim 4, wherein the laser is an excimer laser, a nitrogen laser, or a liquid laser.

6. The method of claim 5, wherein the excimer laser is an ArF laser (193 nm), a KrF laser (248 nm), or a XeCl laser (308 nm).

7. The method of claim 3, wherein the wavelength band X is selected from wavelength regions of 1100-1200 nm, 1350-1450 nm, 1500-1800 nm, or 2100 nm or more.

8. The method of claim 7, wherein the laser is a semiconductor laser, an argon laser, a glass laser, a titanium-sapphire laser, or a fiber laser.

9. A method for writing wafer case information on a given area of a wafer case by irradiating laser lights, wherein the laser lights are irradiated on a same area of a surface of the wafer case from two or more different directions to the wafer case surface.

10. The method of claim 9, wherein an energy of each laser light for heating the wafer case is smaller than an energy for melting a material of the wafer case, and a summation of an energy of each laser light for heating the wafer case is larger than an energy for melting the wafer case material.

11. The method of claim 9, wherein an energy of each laser light for heating the wafer case is smaller than an energy for evaporating a material of the wafer case, and a summation of an energy of each laser light for heating the wafer case is larger than an energy for evaporating the wafer case material.

12. The method of claim 9, wherein an energy of each laser light for heating the wafer case is smaller than an energy for blackening a material of the wafer case, and a summation of an energy of each laser light for heating the wafer case is larger than an energy for blackening the wafer case material.

13. The method of claim 9, wherein an energy of each laser light for heating the wafer case is smaller than an energy for foaming a material of the wafer case, and a summation of an energy of each laser light for heating the wafer case is larger than an energy for foaming the wafer case material.

14. The method of claim 9, wherein a visible light transmittance of a material of the wafer case is 80% or more.

15. The method of claim 10, wherein a visible light transmittance of a material of the wafer case is 80% or more.

16. The method of claim 11, wherein a visible light transmittance of a material of the wafer case is 80% or more.

17. The method of claim 12, wherein a visible light transmittance of a material of the wafer case is 80% or more.

18. The method of claim 13, wherein a visible light transmittance of a material of the wafer case is 80% or more.

19. The method of claim 9, wherein the laser light has a wavelength of a visible wavelength region.

20. The method of claim 10, wherein the laser light has a wavelength of a visible wavelength region.

21. The method of claim 11, wherein the laser light has a wavelength of a visible wavelength region.

22. The method of claim 12, wherein the laser light has a wavelength of a visible wavelength region.

23. The method of claim 13, wherein the laser light has a wavelength of a visible wavelength region.
